(19) United States
(12) Patent Application Publication
(10) Pub. No.: US 2004/0127465 A1
(43) Pub. Date: Jul. 1, 2004

(54) Novel phosphorus-containing derivatives

(75) Inventors: Matthew F. Brown, Stonington, CT (US); Matthew M. Hayward, Old Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/734,411

(22) Filed: Dec. 12, 2003

Related U.S. Application Data
(60) Provisional application No. 60/433,399, filed on Dec. 13, 2002.

(51) Int. Cl. A61K 31/675

(52) U.S. Cl. 514/85; 544/337

(57) ABSTRACT

A compound of the Formula I

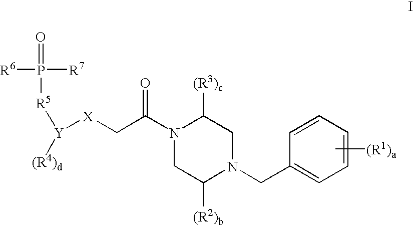

a prodrug thereof, or the pharmaceutically acceptable salt of the compound or prodrug; wherein X, Y, a, b, c, d, R¹, R², R³, R⁴, R⁵, R⁶ and R⁷ are as defined above and are useful to treat inflammation and other immune disorders. The present invention also relates to pharmaceutical compositions that include compounds of Formula I and a pharmaceutically acceptable carrier. Moreover, the present invention relates to methods of using the above-described compounds and compositions to treat and prevent diseases and conditions.

### RELATED APPLICATION

The present application claims priority to U.S. Patent Application Serial No. 60/433,399, filed Dec. 13, 2002, which is incorporated herein in its entirety.

### FIELD OF THE INVENTION

The present invention relates to selective inhibitors of MIP-1α (CCL3) binding to its receptor CCR1, pharmaceutical compositions comprising the compounds and the use of such compounds for treating diseases associated with inflammation and autoimmune disorders.

### BACKGROUND OF THE INVENTION

The compounds of the invention are selective inhibitors of MIP-1α (CCL3) binding to its receptor CCR1 found on inflammatory and immunomodulatory cells (preferably leukocytes and lymphocytes). The CCR1 receptor is sometimes referred to as the CC-CKR1 receptor.

These compounds also inhibit MIP-1α, and the related chemokines shown to interact with CCR1 (e.g., RANTES (CCL5), MCP-2 (CCL8), MCP-3 (CCL7), HCC-1 (CCL14) and HCC-2 (CCL15)), induced chemotaxis of THP-1 cells and human leukocytes and are potentially useful for the treatment or prevention of autoimmune diseases.

MIP-1α and RANTES are soluble chemotactic peptides (chemokines) that are produced by inflammatory cells, in particular CD8+ lymphocytes, polymorphonuclear leukocytes (PMNs) and macrophages, J. Biol. Chem., 270 (30) 29671-29675 (1995). These chemokines act by inducing the migration and activation of key inflammatory and immunomodulatory cells. As reported by Teran, et al., elevated levels of chemokines were found in the synovial fluid of rheumatoid arthritis patients, chronic and rejecting tissue from transplant patients and in the nasal secretions of allergic rhinitis patients following allergen exposure (Teran, et al., J. Immunol., 1806-1812 (1996), and Kuna et al., J. Allergy Clin. Immunol. 321 (1994)).

Antibodies which interfere with the chemokine/receptor interaction by neutralizing MIP1α or gene disruption have provided direct evidence for the role of MIP-1α and RANTES in disease by limiting the recruitment of monocytes and CD8+ lymphocytes (Smith et al., J. Immunol, 153, 4704 (1994) and Cook et al., Science, 269, 1583 (1995)).

The compounds described within are selective antagonists of the CCR1 receptor.

### SUMMARY OF THE INVENTION

In one aspect, the invention is directed to a compound of the Formula I,

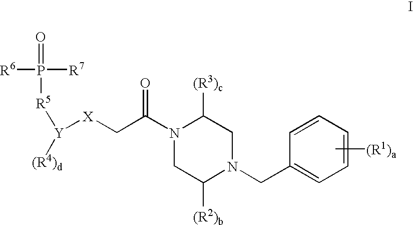

a prodrug thereof, or a pharmaceutically acceptable salt of the compound or the prodrug thereof; wherein,

a=0, 1, 2, 3, 4 or 5;

b=0, 1 or 2;

c=0, 1 or 2;

d=0, 1, 2, 3 or −4;

X is O, S, CH₂ or NR⁶;

Y is (C₆-C₁₀)aryl or (C₂-C₉)heteroaryl;

each R¹ is independently: hydroxy, halo, (C₁-C₈)alkyl optionally substituted with 1 to 3 fluorine atoms, (C₁-C₈)alkoxy optionally substituted with 1-3 fluorine atoms, HO(C₁-C₈)alkyl-, cyano, amino, H₂N(C₁-C₈)alkyl-, carboxy, acyl, (C₁-C₈)alkyl(C═O)(C₁-C₈)alkyl-, H₂N(C═O)—, or H₂N(C═O)(C₁-C₈)alkyl-;

each R² and R³ are independently: oxo, (C₁-C₈)alkyl optionally substituted with 1-3 fluorine atoms, (C₃-C₈)cycloalkyl-, (C₃-C₈)cycloalkyl-(C₁-C₈)alkyl-, (C₆-C₁₀)aryl-, (C₆-C₁₀)aryl(C₁-C₈)alkyl-, HO(C₁-C₈)alkyl-, (C₁-C₈)alkyl-O—(C₁-C₈)alkyl-, H₂N(C₁-C₈)alkyl-, (C₁-C₈)alkyl-NH—(C₁-C₈)alkyl-, [(C₁-C₈)alkyl]₂N—(C₁-C₈)alkyl-, (C₂-C₉)heterocyclyl(C₁-C₈)alkyl-, (C₁-C₈)alkyl(C═O)NH(C₁-C₈)alkyl-, (C₁-C₈)alkyl-O—(C═O) NH(C₁-C₈)alkyl-, H₂N(C═O)NH(C₁-C₈)alkyl-, (C₁-C₈)alkyl-SO₂—NH(C₁-C₈)alkyl-, (C₂-C₉)heteroaryl(C₁-C₈)alkyl-, H₂N(C═O), or H₂N(C═O)(C₁-C₈)alkyl-;

each R⁴ is independently: HO—, halo-, NC—, HO(C═O)—, H₂N—, (C₁-C₈)alkylNH—, [(C₁-C₈)alkyl]₂N—, (C₁-C₈)alkyl-, optionally substituted with 1-3 fluorine atoms, (C₁-C₈)alkoxy optionally substituted with 1-3 fluorine atoms, HO(C₁-C₈)alkyl-, (C₁-C₈)alkyl-O—(C₁-C₈)alkyl-, H₂N(C₁-C₈)alkyl—, (C₁-C₈)alkylNH(C₁-C₈)alkyl-, [(C₁-C₈)alkyl]₂N(C₁-C₈)alkyl-, (C₁-C₈)alkyl(C═O)—, (C₁-C₈)alkyl(C═O)(C₁-C₈)alkyl-, (C₆-C₁₀)aryl-, (C₂-C₉)heteroaryl-, (C₆-C₁₀)aryloxy-, H₂N(C═O)—, H₂N(C═O)(C₁-C₈)alkyl-, (C₁-C₈)alkylNH(C═O)—, (C₁-C₈)alkyl-NH(C═O)(C₁-C₈)alkyl-, [(C₁-C₈)alkyl]₂N(C═O)—, [(C₁-C₈)alkyl]₂N(C═O)(C₁-C₈)alkyl-, (C₃-C₈)cycloalkyl-, (C₁-C₈)alkylSO₂—, NC(C₁-C₈)alkyl-, (C₁-C₈)alkyl(C═O)NH—, H₂N(C═O)NH— or H₂N(C═O)NH(C₁-C₈)alkyl-;

R⁵ is a bond or a (C₁-C₈)alkyl-;

R⁶ is independently: hydroxy, amine or (C₁-C₈)alkyl-NH—; and

R⁷ is independently: hydrogen, hydroxyl, (C₁-C₈)alkoxy- or (C₁-C₈)alkyl-.

In a preferred embodiment, the compound of Formula I has the stereochemistry showin in Formula Ia

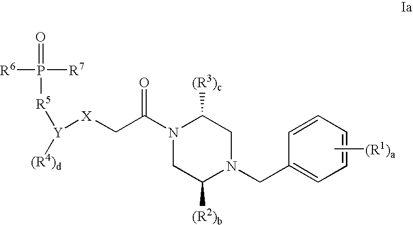

wherein a, b, c, X, Y, R¹, R², R³, R⁴, R⁵, R⁶ and R⁷ are as described above.

In a preferred embodiment, R¹ is: hydroxy, halo, cyano, (C₁-C₈)alkyl-optionally substituted with 1-3 fluorine atoms, or (C₁-C₈)alkoxy optionally substituted with 1-3 fluorine atoms.

In another preferred embodiment, R⁴ is hydroxyl, cyano, (C₁-C₈)alkyl-optionally substituted with 1-3 fluorine atoms, (C₁-C₈)alkoxy optionally substituted with 1-3 fluorine atoms, (C₁-C₈)alkyl(C═O)— or halo-.

In a further preferred embodiment, X is O and R⁵ is (C₁-C₃)alkyl-.

In another preferred embodiment, R² and R³ are each independently (C₁-C₈)alkyl-, optionally substituted with 1-3 fluorine atoms; or (C₃-C₈)cycloalkyl-.

In another preferred embodiment, R⁴ is HO—, NC—, (C₁-C₈)alkyl-optionally substituted with 1-3 fluorine atoms, (C₁-C₈)alkoxy optionally substituted with 1-3 fluorine atoms, (C₁-C₈)alkyl(C═O)— or halo-.

In preferred embodiment, X is O and R⁵ is (C₁-C₃)alkyl-.

In another embodiment, R² and R³ are each independently: (C₁-C₈)alkyl-, optionally substituted with 1-3 fluorine atoms; (C₃-C₈)cycloalkyl-; (C₃-C₈)cycloalkyl-(C₁-C₈)alkyl-; (C₆-C₁₀)aryl-; (C₆-C₁₀)aryl(C₁-C₈)alkyl-; HO(C₁-C₈)alkyl-; H₂N(C₁-C₈)alkyl-; (C₂-C₉)heterocyclyl(C₁-C₈)alkyl-; (C₁-C₈)alkyl-O—(C═O)NH(C₁-C₈)alkyl-; H₂N(C═O)NH(C₁-C₈)alkyl-; (C₁-C₈)alkyl-SO₂NH(C₁-C₈)alkyl-; (C₂-C₉)heteroaryl(C₁-C₈)alkyl-; H₂N(C═O)— or H₂N(C═O)(C₁-C₈)alkyl-.

In a preferred embodiment,

R¹ is: HO—, halo-, NC—, (C₁-C₈)alkyl-optionally substituted with 1-3 fluorine atoms, or (C₁-C₈)alkoxy-optionally substituted with 1-3 fluorine atoms;

R² and R³ are each independently (C₁-C₈)alkyl-, optionally substituted with 1-3 fluorine atoms; or (C₃-C₈)cycloalkyl-;

R⁴ is HO—, NC—, (C₁-C₈)alkyl-optionally substituted with 1-3 fluorine atoms, (C₁-C₈)alkoxy optionally substituted with 1-3 fluorine atoms, (C₁-C₈)alkyl(C═O)— or halo-;

X is O; and

R⁵ is (C₁-C₃)alkyl-.

In another preferred embodiment, the compound of Formula I is:

(5-Chloro-2-{2-[4-(4-fluoro-benzyl)-(2R,5S)-2,5-dimethyl-piperazin-1-yl]-2-oxo-ethoxy}-benzyl)-phosphonic acid;

(5-Chloro-2-{2-[4-(4-fluoro-benzyl)-(2R)-2-methyl-piperazin-1-yl]-2-oxo-ethoxy}-benzyl)-phosphonic acid;

(5-Chloro-2-{2-[(2R)-2-ethyl-4-(4-fluoro-benzyl)-piperazin-1-yl]-2-oxo-ethoxy}-benzyl)-phosphonic acid;

(5-Bromo-2-{2-[4-(4-fluoro-benzyl)-(2R,5S)-2,5-dimethyl-piperazin-1-yl]-2-oxo-ethoxy}benzyl)-phosphonic acid;

(5-Bromo-2-{2-[4-(4-fluoro-benzyl)-(2R)-2-methyl-piperazin-1-yl]-2-oxo-ethoxy}benzyl)-phosphonic acid;

[2-(5-Chloro-2-{2-[4-(4-fluoro-benzyl)-(2R,5S)-2,5-dimethyl-piperazin-1-yl]-2-oxo-ethoxy}-phenyl)-ethyl]-phosphonic acid;

[2-(5-Chloro-2-{2-[4-(4-fluoro-benzyl)-(2R)-2-methyl-piperazin-1-yl]-2-oxo-ethoxy}-phenyl)-ethyl]-phosphonic acid;

[2-(5-Bromo-2-{2-[4-(4-fluoro-benzyl)-(2R,5S)-2,5-dimethyl-piperazin-1-yl]-2-oxo-ethoxy}-phenyl)-ethyl]-phosphonic acid;

[2-(5-Bromo-2-{2-[4-(4-fluoro-benzyl)-(2R)-2-methyl-piperazin-1-yl]-2-oxo-ethoxy}-phenyl)-ethyl]-phosphonic acid;

(5-Chloro-2-{2-[4-(4-chloro-benzyl)-(2R,5S)-2,5-dimethyl-piperazin-1-yl]-2-oxo-ethoxy}-benzyl)-phosphonic acid;

(5-Chloro-2-{2-[4-(4-chloro-benzyl)-(2R)-2-methyl-piperazin-1-yl]-2-oxo-ethoxy}-benzyl)-phosphonic acid;

(5-Bromo-2-{2-[4-(4-chloro-benzyl)-(2R,5S)-2,5-dimethyl-piperazin-1-yl]-2-oxo-ethoxy}-benzyl)-phosphonic acid;

(5-Bromo-2-{2-[4-(4-chloro-benzyl)-(2R)-2-methyl-piperazin-1-yl]-2-oxo-ethoxy}-benzyl)-phosphonic acid;

(5-Chloro-2-{2-[4-(3,4-difluoro-benzyl)-(2R,5S)-2,5-dimethyl-piperazin-1-yl]-2-oxo-ethoxy}-benzyl)-phosphonic acid;

(5-Chloro-2-{2-[4-(3,4-difluoro-benzyl)-(2R)-2-methyl-piperazin-1-yl]-2-oxo-ethoxy}-benzyl)-phosphonic acid;

(5-Bromo-2-{2-[4-(3,4-difluoro-benzyl)-(2R,5S)-2,5-dimethyl-piperazin-1-yl]-2-oxo-ethoxy}-benzyl)-phosphonic acid;

(5-Bromo-2-{2-[4-(3,4-difluoro-benzyl)-(2R)-2-methyl-piperazin-1-yl]-2-oxo-ethoxy}-benzyl)-phosphonic acid;

[2-(5-Chloro-2-{2-[4-(4-chloro-benzyl)-(2R,5S)-2,5-dimethyl-piperazin-1-yl]-2-oxo-ethoxy}-phenyl)-ethyl]-phosphonic acid;

[2-(5-Bromo-2-{2-[4-(4-chloro-benzyl)-(2R,5S)-2,5-dimethyl-piperazin-1-yl]-2-oxo-ethoxy}-phenyl)-ethyl]-phosphonic acid;

[2-(5-Chloro-2-{2-[4-(3,4-difluoro-benzyl)-(2R,5S)-2,5-dimethyl-piperazin-1-yl]-2-oxo-ethoxy}-phenyl)-ethyl]-phosphonic acid;

[2-(5-Bromo-2-{2-[4-(3,4-difluoro-benzyl)-(2R,5S)-2,5-dimethyl-piperazin-1-yl]-2-oxo-ethoxy}-phenyl)-ethyl]-phosphonic acid;

(5-Chloro-2-{2-[4-(4-fluoro-benzyl)-(2R,5S)-2,5-dimethyl-piperazin-1-yl]-2-oxo-ethoxy}pyridin-3-ylmethyl)-phosphonic acid;

(5-Bromo-2-{2-[4-(4-fluoro-benzyl)-(2R,5S)-2,5-dimethyl-piperazin-1-yl]-2-oxo-ethoxy}-pyridin-3-ylmethyl)-phosphonic acid;

[2-(5-Chloro-2-{2-[4-(4-fluoro-benzyl)-(2R,5S)-2,5-dimethyl-piperazin-1-yl]-2-oxo-ethoxy}-pyridin-3-yl)-ethyl]-phosphonic acid;

[2-(5-Bromo-2-{2-[4-(4-fluoro-benzyl)-(2R,5S)-2,5-dimethyl-piperazin-1-yl]-2-oxo-ethoxy}-pyridin-3-yl)-ethyl]-phosphonic acid;

(5-Chloro-2-{2-[4-(4-fluoro-benzyl)-(2R,5S)-2,5-dimethyl-piperazin-1-yl]-2-oxo-ethoxy}-benzyl)-phosphinic acid;

(5-Chloro-2-{2-[4-(4-fluoro-benzyl)-(2R,5S)-2,5-dimethyl-piperazin-1-yl]-2-oxo-ethoxy}-benzyl)-methyl-phosphinic acid;

(5-Chloro-2-{2-[4-(4-fluoro-benzyl)-(2R,5S)-2,5-dimethyl-piperazin-1-yl]-2-oxo-ethoxy}-benzyl)-ethyl-phosphinic acid;

(5-Chloro-2-{2-[4-(4-fluoro-benzyl)-(2R,5S)-2,5-dimethyl-piperazin-1-yl]-2-oxo-ethoxy}-benzyl)-phosphonic acid monomethyl ester;

(5-Chloro-2-{2-[4-(4-fluoro-benzyl)-(2R,5S)-2,5-dimethyl-piperazin-1-yl]-2-oxo-ethoxy}-benzyl)-phosphonic acid monoethyl ester;

(5-Chloro-2-{2-[4-(4-fluoro-benzyl)-(2R,5S)-2,5-dimethyl-piperazin-1-yl]-2-oxo-ethoxy}-benzyl)-ethyl-phosphonamidic acid;

(5-Chloro-2-{2-[4-(4-fluoro-benzyl)-(2R,5S)-2,5-dimethyl-piperazin-1-yl]-2-oxo-ethoxy}-benzyl)-phosphonamidic acid monomethyl ester; or

(5-Chloro-2-{2-[4-(4-fluoro-benzyl)-(2R,5S)-2,5-dimethyl-piperazin-1-yl]-2-oxo-ethoxy}-benzyl)-phosphonamidic acid monoethyl ester.

In a second aspect, the invention is directed to a pharmaceutical composition comprising a therapeutically effective amount of a compound as described above, a prodrug thereof or a pharmaceutically acceptable salt of the compound or the prodrug, and a pharmaceutically acceptable diluent or carrier.

In a third aspect, the invention is directed to a therapeutic method of inhibiting MIP-1α and/or RANTES from binding to the receptor CCR1 in a mammal, including a human, comprising administering to a mammal in need of such treatment a therapeutically effective amount of a compound of Formula I.

In a fourth aspect, the invention is directed to a method of treating a condition mediated by inhibiting MIP-1αl and/or RANTES from binding to the receptor CCR1, comprising admininistering to a mammal in need of such treatment a therapeutically effective amount of a compound of Formula I.

In a preferred embodiment, the the condition treated or prevented is selected from autoimmune diseases; fibrosis, allergic conditions, acute and chronic lung inflammation, atherosclerosis, Alzheimer's disease, vascular inflammation resulting from tissue transplant or during restenosis, acute and chronic inflammatory conditions, acute or chronic transplant rejection, HIV infectivity, granulomatous diseases, conditions associated with leptin production, sequelae associated with cancer, tissue damage caused by inflammation induced by infectious agents, viral inflammation of the lung or liver, gastrointestinal inflammation, or inflammation resulting from bacterial meningitis, HIV-1, HIV-2, HIV-3, cytomegalovirus, adenoviruses, Herpes viruses, fungal meningitis, lyme disease, or malaria.

In a further preferred embodiment, the condition is selected from the group consisting of rheumatoid arthritis; Takayasu arthritis; psoriatic arthritis; ankylosing spondylitis; type I diabetes (recent onset); lupus; inflammatory bowel disease; Chrohn's disease; optic neuritis; psoriasis; multiple sclerosis; polymyalgia rheumatica; uveitis; thyroiditis; vasculitis; pulmonary fibrosis; idiopathic pulmonary fibrosis; interstitial pulmonary fibrosis; fibrosis associated with end-stage renal disease; fibrosis caused by radiation; tubulointerstitial fibrosis; subepithelial fibrosis; scleroderma; progressive systemic sclerosis; hepatic fibrosis; primary and secondary biliary cirrhosis; asthma; contact dermatitis; atopic dermatitis; chronic bronchitis; chronic obstructive pulmonary disease; adult Respiratory Distress Syndrome; Respiratory Distress Syndrome of infancy; immune complex alveolitis; restenosis following angioplasty and/or stent insertion; synovial inflammation caused by arthroscopy, hyperuremia, or trauma; osteoarthritis; ischemia reperfusion injury; glomerulonephritis; nasal polyosis; enteritis; Behcet's disease; preeclampsia; oral lichen planus; Guillian-Barre syndrome; xeno-transplantation rejection; sarcoidosis; leprosy; tuberculosis; obesity; cachexia; anorexia; type II diabetes; hyperlipidemia; hypergonadism; sequelae associated with multiple myeloma; viral-induced encephalomyelitis or demyelination; viral inflammation of the lung or liver caused by influenza or hepatitis; and H. pylori infection.

In a fifth aspect, the invention is directed to a therapeutic method of treating a condition mediated by inhibiting the production of metalloproteinases and cytokines at inflammatory sites comprising administering to a mammal, including a human, in need of such treatment a therapeutically effective a mount of a compound of Formula I.

In a preferred embodiment, the inflammatory site is MMP9, TNF, IL-1 or IL-6.

In further preferred embodiment, the condition treated is joint tissue damage, hyperplasia, pannus formation, bone resorption, hepatic failure, Kawasaki syndrome, myocardial infarction, acute liver failure, septic shock, congestive heart failure, pulmonary emphysema or dyspnea associated therewith.

In a sixth aspect, the invention is directed to a therapeutic method of antagonizing the CCR1 receptor in a mammal, including a human, comprising administering to a mammal in need of such treatment a therapeutically effective amount of a compound of Formula I.

In a seventh aspect, the invention is directed to a pharmaceutical composition that comprises a therapeutically effect amount of an inhibitor of MIP-1α and/or RANTES binding to the receptor CCR1, according to the compound of Formula I as described above; and at least one of the following: Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778, FTY-720, BMS-188667, RG-1046, Prednisone, Prednisolone, Methylprednisolone suleptanate, Cortisone, Hydrocortisone, Methotrexate, Sulfasalazine, Etanercept, Infliximab, Adalimumab, CDP-571, Anakinra, NSAIDS, Celecoxib, Valdecoxib, Rofecoxib, Anti-interleukin-6 receptor monoclonal antibody, Glatiramer acetate, Interferon beta 1-a, Interferon beta 1-b, Mitoxantrone, Pimecrolimus or agents that inhibit cell recruitment mechanisms.

The term(s) “compound(s) of Formula I” and “compound(s) of this invention” as used herein, means a compound or compounds of Formula I, prodrugs thereof and pharmaceutically acceptable salts of the compounds or the prodrugs. The term “compound(s),” when referring to compounds of Formula I, also includes prodrugs of the compound(s) and pharmaceutically acceptable salts of the compound(s) or the prodrugs.

The expression “pharmaceutically acceptable salt” as used herein in relation to compounds of Formula I of this invention includes pharmaceutically acceptable anionic salts. The term “pharmaceutically acceptable anion” refers to a negative ion that is compatible chemically and/or toxicologically with the other ingredients of a pharmaceutical composition and/or the animal being treated therewith. Suitable anions include, but are not limited to, halides (e.g., chloride, iodide, and bromide), (C₁-C₁₂)alkylsulfonates (e.g., mesylate, ethylsulfonate, etc.), arylsulfonates (e.g., phenylsulfonate, tosylate, etc.), (C₁-C₁₂)alkylphosphonates, di(C₁-C₁₂)alkylphosphates (e.g., dimethylphosphate, diethylphosphate, α-diglycerol phosphate, etc.), arylphosphonates, arylphosphates, alkylarylphosphonates, alkylarylphosphates, (C₁-C₁₂)alkylcarboxylates (e.g., acetates, propionates, glutamates, glycerates, etc.), arylcarboxylates, and the like.

The compounds of the present invention may be isolated and used per se or in the form of its pharmaceutically acceptable salt, solvate and/or hydrate. The term “salts” refers to inorganic and organic salts of a compound of the present invention. These salts can be prepared in situ during the final isolation and purification of a compound, or by separately reacting the compound, or prodrug with a suitable organic or inorganic acid and isolating the salt thus formed. Representative salts include the hydrobromide, hydrochloride, hydroiodide, sulfate, bisulfate, nitrate, acetate, trifluoroacetate, oxalate, besylate, palmitiate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine; and the like. See, e.g., Berge, et al., J. Pharm. Sci., 66, 1-19 (1977).

The term “prodrug” means a compound that is transformed in vivo to yield a compound of Formula (I) or a pharmaceutically acceptable salt, hydrate or solvate of the compound. The transformation may occur via various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

Those skilled in the art will further recognize that the compounds of Formula I can exist in crystalline form as hydrates wherein molecules of water are incorporated within the crystal structure thereof and as solvates wherein molecules of a solvent are incorporated therein. All such hydrate and solvate forms are considered part of this invention.

This invention also includes isotopically-labeled compounds, which are identical to those described by Formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, sulfur, and fluorine, such as ²H, ³H, ¹³C, ¹⁴C, ¹⁵N, ¹⁸O, ¹⁷O, and ¹⁸F, respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of Formula I of this invention and prodrugs thereof can generally be prepared by carrying out the procedures disclosed in the schemes and/or in the Examples below, by substituting a readily available isotopically labeled reagent for a non-isotopically labeled reagent.

The compounds of this invention may contain olefin-like double bonds. When such bonds are present, the compounds of the invention exist as cis and trans configurations and as mixtures thereof.

The term “alkyl” as used herein, unless otherwise indicated, means a saturated monovalent straight or branched aliphatic hydrocarbon radical that may also be cyclic (e.g., cyclopropyl, cyclobutyl, cyclopentyl, cyclohexyl, cycloheptyl) or bicyclic (e.g., norbornanyl, bicyclo[3.2.1]octane) or contain cyclic groups. The term “alkyl” also zero to two levels of unsaturation. The alkyl groups may also be optionally substituted with 1 to 3 substituents. Examples of substitutents independently selected include, but are not limited to: halo-, HO—, NC—, H₂N—, HO—(C═O)—.

Unless otherwise indicated, halogen includes fluorine, chlorine, bromine, and iodine.

The term “(C₂-C₉)Heterocyclyl-” when used herein refers to, but is not limited to, pyrrolidinyl, tetrahydrofuranyl, dihydrofuranyl, tetrahydropyranyl, pyranyl, thiopyranyl, aziridinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl and chromanyl. Said (C₂-C₉)heterocyclyl ring is attached through a carbon or a nitrogen atom.

The term “(C₂-C₉)Heteroaryl”, when used herein, refers to, but is not limited to, furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, and may be optionally substituted with 1 to 3 substituents independently selected from the group consisting of, but not limited to: H—, HO—, halo-, (C₁-C₈)alkyl-optionally substituted with 1-3 fluorine atoms, (C₁-C₈)alkyl-O— wherein the alkyl group is optionally substituted with 1-3 fluorine atoms, HO—(C₁-C₈)alkyl-, NC—, H₂N—, H₂N(C₁-C₈)alkyl-, HO(C═O)—, (C₁-C₈)alkyl(C═O)—, (C₁-C₈)alkyl(C═O)(C₁-C₈)alkyl-, H₂N(C═O)—, H₂N(C═O)(C₁-C₈)alkyl-, H₂NSO₂— or (C₁-C₈)alkyl-SO₂—NH—.

The term “aryl”, when used herein, refers to phenyl or naphthyl that may independently be optionally substituted with 1 to 3 substituents. Examples of substitutents include, but are not limited to, H—, HO—, halo-, (C₁-C₈)alkyl-optionally substituted with 1-3 fluorine atoms, (C₁-C₈)alkoxy optionally substituted with 1-3 fluorine atoms, HO(C₁-C₈)alkyl-, NC—, H₂N—, H₂N(C₁-C₈)alkyl-, HO(C═O)—, (C₁-C₈)alkyl(C═O)—, (C₁-C₈)alkyl(C═O)(C₁-C₈)alkyl-, H₂N(C═O)—, H₂N(C═O)(C₁-C₈)alkyl-, H₂NSO₂— or (C₁-C₈)alkylSO₂NH—.

The compounds of this invention include all tautomers, conformational isomers (e.g., cis and trans isomers) and all optical isomers of compounds of the Formula I (e.g., enantiomers and diastereomers), as well as racemic, diastereomeric and other mixtures of such isomers. Some of the compounds described herein contain at least one stereogenic center; consequently, those skilled in the art will appreciate that all stereoisomers (e.g., enantiomers and diasteroisomers, and racemic mixtures thereof) of the compounds illustrated and discussed herein are within the scope of the present invention.

The compounds of the invention are useful for the treatment or prevention of autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; Alzheimer's disease; vascular inflammation resulting from tissue transplant or during restenosis (including, but not limited to, restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); and sequelae associated with certain cancers such as multiple myeloma.

This method of treatment may also have utility for the prevention of cancer metastasis, including but not limited to, breast cancer.

This method of treatment may also inhibit the production of metalloproteinases and cytokines at inflammatory sites (including but not limited to, MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith).

This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (e.g. resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease or malaria).

### DETAILED DESCRIPTION OF THE INVENTION

The compounds of the invention are selective inhibitors of MIP-1αl (CCL3) binding to its receptor CCR1 found on inflammatory and immunomodulatory cells (preferably leukocytes and lymphocytes). These compounds also inhibit MIP-1α, and the related chemokines shown to interact with CCR1 (e.g., RANTES (CCL5), MCP-2 (CCL8), MCP-3 (CCL7), HCC-1 (CCL14) and HCC-2 (CCL15)), induced chemotaxis of THP-1 cells and human leukocytes.

In general, the compounds of Formula I of this invention may be prepared by methods that include processes known in the chemical arts, particularly in light of the description contained herein. Certain processes for the manufacture of the compounds of Formula I of this invention are illustrated by the following reaction schemes. Other processes are described in the experimental section. Some of the starting compounds for the reactions described in the schemes and examples are prepared as illustrated in Preparation A and Preparation B. All other starting compounds may be obtained from general commercial sources, such as Sigma-Aldrich Corporation, St. Louis, Mo.

The following reaction Schemes illustrate the preparation of the compounds of the present invention. Preparation A and Preparation B schemes depict the preparation of starting compounds for Schemes 1 and 2. Unless otherwise indicated, a, b, c, and d, as well as R¹ through R⁷, are defined as above.

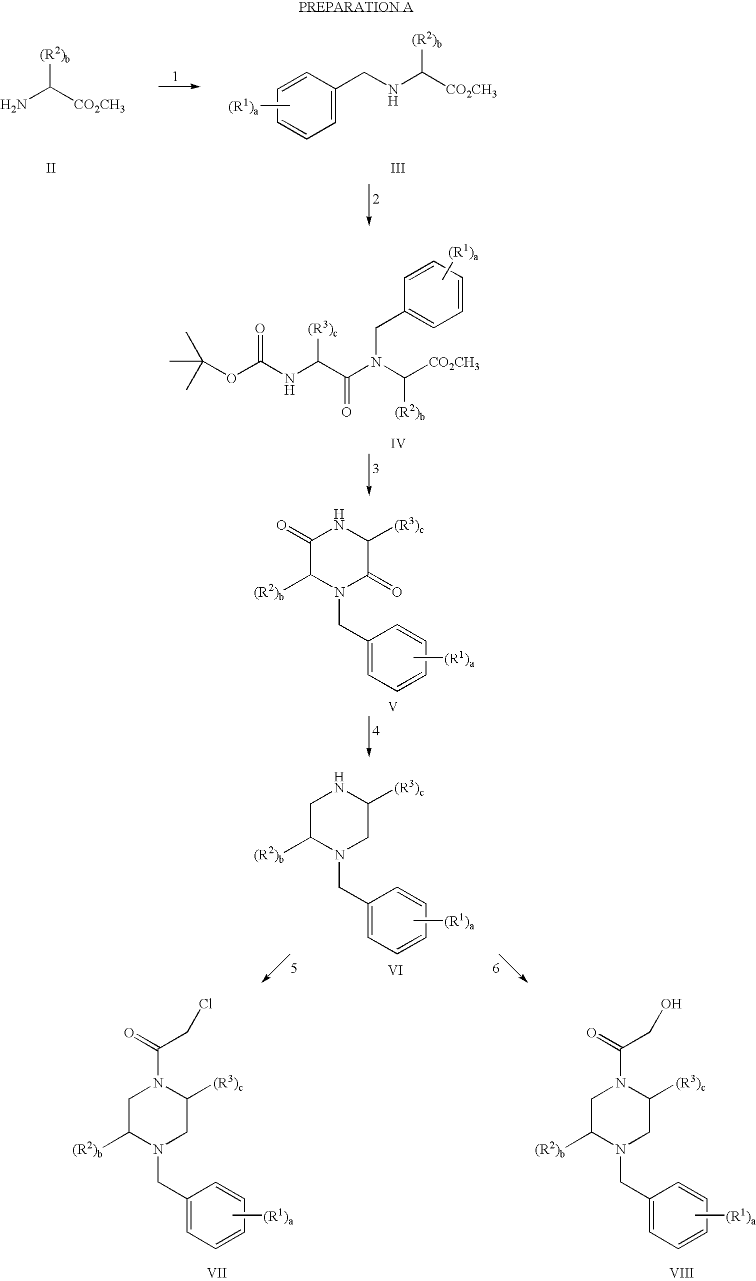

In reaction 1 of Preparation A, the compound of Formula II, wherein b is 0, 1 or 2, may be converted to the corresponding compound of Formula III by reacting II with a benzaldehyde compound of the Formula

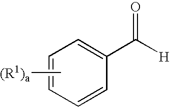

in the presence of a base, such as triethylamine, and a reducing agent, such as sodium triacetoxyborohydride, in an aprotic solvent, such as 1,2-dichloroethane. The reaction mixture is stirred at room temperature for a time period between about 1 hour to about 4 hours, preferably about 2 hours.

In reaction 2 of Preparation A, the compound of Formula III may be converted to the corresponding compound of Formula IV by first reacting a compound of the Formula

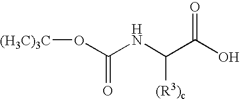

wherein c is 0, 1 or 2, with 4-methyl morpholine and isobutylchloroformate in the presence of a polar aprotic solvent, such as tetrahydrofuran, followed by reacting the intermediate so formed with the compound of Formula III. The reaction mixture is stirred overnight at ambient temperature.

In reaction 3 of Preparation A, the compound of Formula IV may then be converted to the corresponding piperizine-2,5-dione compound of Formula V by treating IV with trifluoroacetic acid in the presence of a polar aprotic solvent, such as methylene chloride. The reaction is stirred at room temperature for a time period between about 1 hour to about 4 hours, preferably about 2 hours.

In reaction 4 of Preparation A, the compound of Formula V may be converted to the corresponding compound of Formula VI by reducing V with a reducing agent, such as lithium aluminum hydride. The reaction is conducted at a temperature between about −10° C. to about 10° C., preferably about 0° C., for a time period between about 10 minutes to about 90 minutes, preferably about 40 minutes.

In reaction 5 of Preparation A, the compound of Formula VI may be converted to the corresponding compound of Formula VII by reacting compound VI with chloroacetyl chloride in the presence of a base, such as triethylamine, in a polar aprotic solvent, such as methylene chloride, at ambient temperature for a time period between 15 minutes and 3 hours, preferably about 30 minutes.

In reaction 6 of Preparation A, the compound of Formula VI may be converted to the corresponding compound of Formula VIII by reacting VI with acetoxy acetylchloride in the presence of a base, such as triethylamine, in a polar aprotic solvent, such as methylene chloride, at ambient temperature for a time period between 15 minutes and 4 hours, preferably about 1 hour. The resulting acetyl-protected alcohol is then be reacted with lithium hydroxide hydrate in a solvent mixture including water, tetrahydrofuran and methanol, at ambient temperature for a time period between 1 hour and 8 hours, preferably about 2 hours.

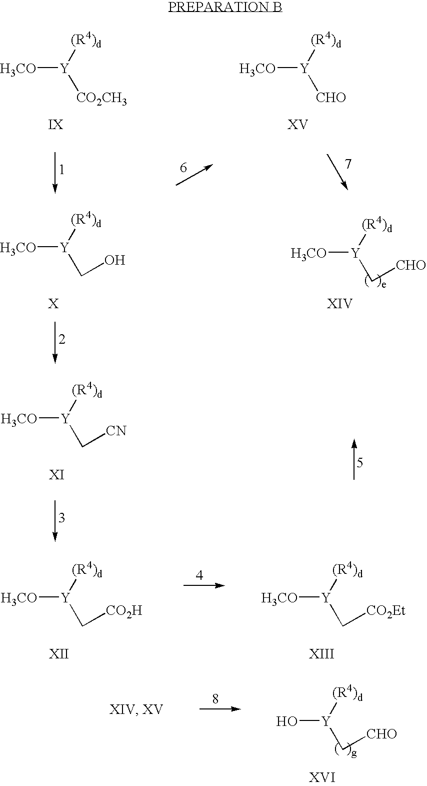

In reaction 1 of Preparation B, the compound of Formula IX is converted to the corresponding compound of the Formula X by treating IX with a reducing agent, such as lithium aluminum hydride, in an aprotic solvent, such as tetrahydrofuran. The reaction mixture is heated to reflux for a time period between 1 hour and 6 hours, preferably about 2 hours.

In reaction 2 of Preparation B, the compound of Formula X is converted to the corresponding compound of the Formula XI by first converting the hydroxyl group to a chloro group by reacting X with thionyl chloride, in the presence of an aprotic solvent, such as methylene chloride. The reaction is heated to reflux, for a time period between about 1 hour to about 10 hours, preferably about 3 hours. The resulting alkyl chloride is then treated with a cyanide source, such as potassium cyanide, in the presence of an aprotic solvent, such as acetonitrile and a crown ether, such as 18-crown-6. The reaction mixture is stirred at ambient temperature for a time period between about 1 hour to about 10 hours, preferably about 3 hours.

In reaction 3 of Preparation B, the compound of Formula XI is converted to the compound of Formula XII by first treating XI with a hydroxide source, such as potassium hydroxide in a mixture of ethanol and water. The reaction mixture is heated to reflux for a time period between about 1 hour to about 10 hours, preferably about 8 hours.

In reaction 4 of Preparation B, the compound of Formula XII is converted to the compound of Formula XIII by treating with ethanol in the presence of an acid, such as hydrochloric acid, at ambient temperature for a time period between about 8 hours to about 16 hours, preferably about 12 hours.

In reaction 5 of Preparation B, the compound of Formula XIII is converted to the corresponding compound of Formula XIV, wherein e is 1, by first treating XIII with an reducing agent, as analogously described above in reaction 1 of Preparation B. The resultant alcohol is converted to XIV with an oxidizing agent, such as Dess-Martin periodinane, in the presence of an aprotic solvent, such as tetrahydrofuran at ambient temperature for a time period between about 1 hour to about 16 hours, preferably about 4 hours.

In reaction 6 of Preparation B, the compound of Formula X is converted to the corresponding compound of Formula XV by first treating X with an oxidizing agent, such as Dess-Martin periodinane, in the presence of an aprotic solvent, such as tetrahydrofuran at ambient temperature for a time period between about 1 hour to about 16 hours, preferably about 4 hours.

In reaction 7 of Preparation B, the compound of Formula XV is converted to the corresponding compound of Formula XIV, wherein e is 2-7, by first treating XV with a phosphonium ylide derived from the phosphonium salt of the Formula:

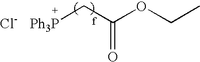

wherein f is 1 to 6, wherein alkyl is defined as above, in the presence of an aprotic solvent, such as tetrahydrofuran. The reaction is conducted at a temperature between −78° C. and reflux. The preferred temperature is dependent on which phosphonium ylide is utilized The reaction is allowed to proceed for a time period between about 4 hours to about 16 hours, preferably about 10 hours (For similar transformations, see: J. Am. Chem. Soc. 1985, 107, 217, incorporated herein by reference in its entirety). The resulting olefinic ester may then hydrogenated by shaking under a positive pressure of hydrogen in the presence of a catalyst, such as platinum dioxide, in the presence of an aprotic solvent, such as ethyl acetate. The ester may then be reduced and reoxidized according to the procedure analogously described above in reaction 5 of Preparation B to afford compound of Formula XIV.

In reaction 8 of Preparation B, compounds of Formula XIV or XV is converted to the corresponding compound of Formula XVI, wherein g is 0 to 7, by demethylating the methyl ether with an acid, such as 47% aqueous hydrogen bromide. The reaction mixture is heated to reflux for a time period between about 10 hours to about 30 hours, preferably about 24 hours.

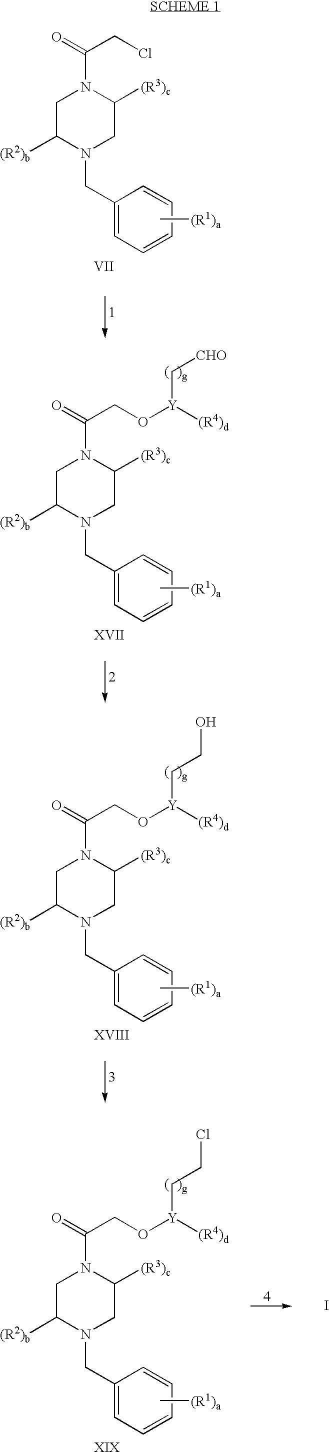

In reaction 1 of Scheme 1, the compound of Formula VII (from Preparation A) is converted to the corresponding compound of Formula XVII, wherein g is 0-7, by reacting VII with a compound of the Formula XVI (from Preparation B) in the presence of potassium carbonate, potassium iodide and an aprotic solvent, such as dimethylformamide. The reaction may be heated to reflux for a time period between about 4 hours to about 8 hours, preferably about 6 hours.

In reaction 2 of Scheme 1, the compound of Formula XVII may be converted to the corresponding compound of Formula XVIII, wherein g is 0-7, by reacting XVII with a reducing agent, such as sodium borohydride, in an aprotic solvent, such as tetrahydrofuran, at a temperature between about −10° C. and ambient temperature, preferably ambient, for a time period between 15 minutes and 90 minutes, preferably about 60 minutes.

In reaction 3 of Scheme 1, the compound of Formula XVIII may be converted to the corresponding compound of Formula XIX, wherein g is 0 to 7, as analogously described above in reaction 2 of preparation B.

In reaction 4 of Scheme 1, the compound of Formula XIX may be converted to the corresponding compound of Formula I by reacting XIX with a phosphate, such as neat trialkylphosphite (e.g. triethylphosphite), at a temperature between 70° C. and 150° C., preferably 130° C. for a time period between 3 and 24 hours, preferably about 12 hours. The diethylphosphonate so formed may then be reacted with trimethylsilyl bromide and anisole in an aprotic solvent, such as methylene chloride, at ambient temperature for a time period between 1 and 12 hours, preferably about 3 hours, thus generating the compound of Formula I.

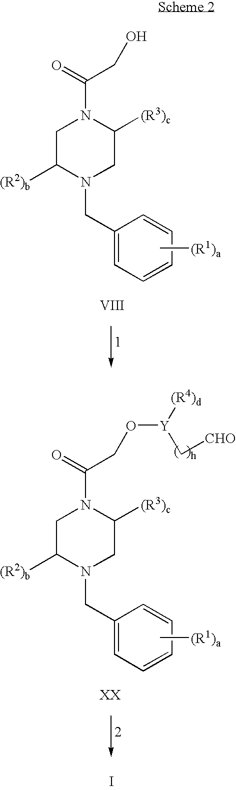

In reaction 1 of Scheme 2, the compound of Formula VIII (from Preparation A) is converted to the corresponding compound of Formula XX by reacting VIII with a compound of Formula

Cl—Y[(R⁴)_{d}]—(CH₂)ₕ—CHO

wherein Y is a (C₂-C₉) heteroaryl, wherein the chlorine is attached to a carbon atom that is adjacent to a heteroatom (for example, 2-pyridyl) and wherein h is 0 to 7. The reactants are stirred in a polar aprotic solvent, such as acetonitrile, in the presence of a base, such as triethylamine, at reflux temperature for a time period between about 4 hours and 24 hours, preferably about 12 hours.

In reaction 2 of Scheme 2, the compound of Formula XX, wherein Y is a (C₂-C₉) heteroaryl, may be converted to the corresponding compounds of Formula I using the methodologies analogously described above in reactions 2-4 of Scheme 1.

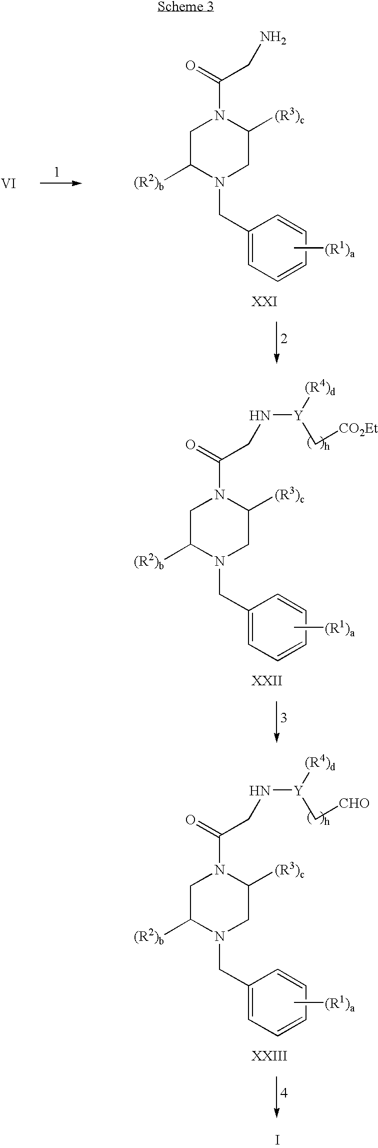

In reaction 1 of Scheme 3, the compound of Formula VI is converted to the corresponding compound of Formula XXI, wherein Y is a (C₂-C₉) heteroaryl, by reacting VI with tert-butoxycarbonylamino-acetic acid in an aprotic solvent, such as methylene chloride, with a carbodiimide, such as dicyclohexylcarbodiimide, in the presence of a base, such as triethylamine, at room temperature for a time period between about 1 and 24 hours, preferably about 3 hours. The compound of Formula XXI may subsequently be produced from this carbamate by the reaction of trifluoroacetic acid at room temperature for a time period between about 1 and 12 hours, preferably about 4 hours.

In reaction 2 of Scheme 3, the compound of Formula XXI may be converted to the corresponding compound of Formula XXII, wherein Y is a (C₂-C₉) heteroaryl, following the precedent analogously described above in reaction 1 of Scheme 2.

In reaction 3 of Scheme 3, the compound of Formula XXII may be converted to the corresponding compound of Formula XXIII, wherein Y is a (C₂-C₉) heteroaryl, by first reducing the ester to the corresponding alcohol with a reducing agent, such as sodium borohydride, in tert-butanol and methanol, at a temperature between about 20° C. and reflux, preferably reflux for a time period between 1 hour and 6 hours, preferably about 1 hour. The resultant alcohol is converted to the compound of Formula XXIII by treating with an oxidizing agent, such as Dess-Martin periodinane, in the presence of an aprotic solvent, such as tetrahydrofuran, at ambient temperature for a time period between about 1 hour to about 16 hours, preferably about 4 hours.

In reaction 4 of Scheme 3, the compound of Formula XXIII, wherein Y is a (C₂-C₉) heteroaryl, may be converted into the compound of Formula I using the methodologies analogously described above in reactions 2-4 of Scheme 1.

Unless indicated otherwise, the reactions may be conducted at a pressure of about one to about three atmospheres, preferably at ambient pressure (about one atmosphere).

The compounds of the Formula I that are basic in nature are capable of forming a wide variety of different salts with various inorganic and organic acids. Although such salts must ultimately be pharmaceutically acceptable for administration to animals, it may be desirable to initially isolate a compound of the Formula I from the reaction mixture as a pharmaceutically unacceptable salt. The “unacceptable” salt may then be simply converted back to the free base compound by treatment with an alkaline reagent, followed by subsequent conversion of the free base to a pharmaceutically acceptable acid addition salt. These salts, both acceptable and unacceptable, are within the scope of this invention.

The acid addition salts of the base compounds of this invention may readily be prepared by treating the base compound with a substantially equivalent amount of the chosen mineral or organic acid in an aqueous solvent medium or in a suitable organic solvent such as methanol or ethanol. Upon evaporation of the solvent, a solid salt may be obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate (i.e., 1,1′-methylene-bis-(2-hydroxy-3-naphthoate)) salts.

Those compounds of the Formula I that are also acidic in nature, are capable of forming base salts with various pharmacologically acceptable cations. Examples of such salts include the alkali metal or alkaline-earth metal salts and particularly, the sodium and potassium salts. These salts are all prepared by conventional techniques known to one of ordinary skill in the art.

The chemical bases that may be used as reagents to prepare the pharmaceutically acceptable base salts of this invention are those which form non-toxic base salts with the herein described acidic compounds of Formula I. These non-toxic base salts include, but are not limited to, those derived from such pharmacologically acceptable cations as sodium, potassium, calcium and magnesium, etc. These salts may readily be prepared by treating the corresponding acidic compounds with an aqueous solution containing the desired pharmacologically acceptable cations, and then evaporating the resulting solution to dryness, preferably under reduced pressure. Alternatively, the salts may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Compounds of the Formula I and their pharmaceutically acceptable salts (hereinafter also referred to, collectively, as “the active compounds”) are potent antagonists of the CCR1 receptor. The active compounds are useful in the treatment or prevention of autoimmune diseases (such as rheumatoid arthritis, Takayasu arthritis, psoriatic arthritis, ankylosing spondylitis, type I diabetes (recent onset), lupus, inflammatory bowel disease, Chrohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulointerstitial fibrosis, subepithelial fibrosis, scleroderma: (progressive systemic sclerosis), hepatic fibrosis (including that caused by alcoholic or viral hepatitis), primary and secondary biliary cirrhosis); allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; Alzheimer's Disease; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xeno-transplantation); HIV infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); and sequelae associated with certain cancers such as multiple myeloma.

This method of treatment may also have utility for the prevention of cancer metastasis, including but not limited to breast cancer.

This method of treatment may also inhibit the production of metalloproteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith).

This method of treatment may also prevent tissue damage caused by inflammation induced by infectious agents (such as viral induced encephalomyelitis or demyelination, viral inflammation of the lung or liver (e.g. caused by influenza or hepatitis), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria).

The activity of the compounds of the invention may-be assessed according to procedures known to those of ordinary skill in the art. Examples of recognized methods for determining CCR1-induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

### Chemotaxis Assay

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/mL of bovine serum albumin. Briefly, MIP-1α (Peprotech, Inc., P.O. Box 275, Rocky Hill N.J.) or other test agonists, are placed into the lower chambers of the Boyden chamber. A polycarbonate filter is then applied and the upper chamber fastened. The amount of agonist chosen is that determined to give the maximal amount of chemotaxis in this system (e.g., typically, 1 nM for MIP-1αl should be adequate).

THP-1 cells (ATCC TIB-202), primary human monocytes, or primary lymphocytes, isolated by standard techniques may then be added to the upper chambers in triplicate, together with various concentrations of the test compound. Compound dilutions may be prepared using standard serological techniques and are mixed with cells prior to adding to the chamber. After a suitable incubation period at 37 degrees centigrade (e.g. 3.5 hours for THP-1 cells, 90 minutes for primary monocytes), the chamber is removed, the cells in the upper chamber aspirated, the upper part of the filter wiped, and the number of cells migrating can be determined according to the following method.

For THP-1 cells, the chamber (a 96 well variety manufactured by Neuroprobe) may be centrifuged to push cells off the lower chamber and the number of cells can be quantitated against a standard curve by a color change of the dye fluorocein diacetate. For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotiant is the % inhibition for the compound, that can then be plotted using standard graphics techniques against the concentration of compound used. The 50% inhibition point is then determined using a line fit analysis for all concentrations tested. The line fit for all data points must have a coefficient of correlation (R squared) of >90% to be considered a valid assay.

All of the compounds of the invention illustrated in the following examples had IC₅₀ of less than 10 μM, in the Chemotaxis assay.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or welting agents (e.g., sodium lauryl sulphate). The tablets may be coated by methods well known in the art.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents.

Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention may conveniently be delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas.

In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, providing, for example, 1, 2 or 3 doses each time.

The active agents may be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, incorporated herein in their entirety.

The compounds of the invention can also be utilized in combination therapy with other therapeutic agents such as, including but not limited to, Cyclosporin A, ISAtx247, Rapamycin, Everolimus, FK-506, Azathioprine, Mycophenolate mofetil, Mycophenolic acid, Daclizumab, Basiliximab, Muromonab, Horse anti-thymocyte globulin, Polyclonal rabbit antithymocyte globulin, Leflunomide, FK-778 (MNA-715), FTY-720, BMS-188667 (CTLA4-1 g), RG-1046 (CTLA4-1 g), Prednisone, Prednisolone, Methylprednisolone suleptanate, Cortisone, Hydrocortisone, Methotrexate, Sulfasalazine, Etanercept, Infliximab, Adalimumab (D2E7), CDP-571, CDP-870, Anakinra, NSAIDS (aspirin, acetaminophen, naproxen, ibuprofen, ketoprofen, diclofenac and piroxicam), Celecoxib, Valdecoxib, Rofecoxib, Anti-interleukin-6 receptor monoclonal antibody (MRA), Glatiramer acetate, Interferon beta 1-a, Interferon beta 1-b, Mitoxantrone, Pimecrolimus, or agents that inhibit cell recruitment mechanisms (eg inhibitors of integrin upregulation or function) or alter leukocyte trafficking.

### General Experimental Procedures

Chromatography refers to column chromatography performed using 32-63 mm silica gel and executed under nitrogen pressure (flash chromatography) conditions.

Particle Beam Mass Spectra were recorded on either a Hewlett Packard 5989®), utilizing chemical ionization (ammonium), or a Fisons (or MicroMass) Atmospheric Pressure Chemical Ionization (APCI) platform which uses a 50/50 mixture of acetonitrile/water.

Room or ambient temperature refers to 20-25° C.

All non-aqueous reactions were run under a nitrogen atmosphere for convenience and to maximize yields.

Concentration in vacuo means that a rotary evaporator was used.

The names for the compounds of the invention were created by the Autonom 2.0 PC-batch version from Beilstein Informationssysteme GmbH (ISBN 3-89536-976-4).

Commercial reagents were utilized without further purification.

The following Examples are intended to illustrate particular embodiments of the invention and are not intended to limit the specification, including the claims in any manner.

### EXAMPLE 1

### (5-Chloro-2-{2-[4-(4-fluoro-benzyl)-(2R,5S)-2,5-dimethyl-piperazin-1-yl]-2-oxo-ethoxy}-benzyl)-phosphonic Acid

Step 1: (S)-2-(4-Fluoro-benzylamino)-propionic Acid Methyl Ester.

To a solution of (S)-2-amino-propionic acid methyl ester hydrochloride (25 grams, 179 mmol) and 4-fluorobenzaldehyde (23 mL, 215 mmol) in 1,2-dichloroethane (200 mL) was added triethylamine (25 mL, 179 mmol). The resulting mixture was stirred for about two hours at ambient temperature, followed by addition of sodium triacetoxyborohydride (57 grams, 268 mmol) in four portions. The resulting mixture was stirred overnight at ambient temperature. The reaction was then neutralized with dilute aqueous sodium hydroxide solution and extracted with dichloromethane. The organic layer was dried over magnesium sulfate, filtered and concentrated in vacuo. Chromatography on silica gel provided the title compound (34.4 g).

Step 2: (2S)-2-[(2R)-(2-tert-Butoxycarbonylamino-propionyl)-(4-fluoro-benzyl)-amino]-propionic Acid Methyl Ester.

To a solution of (R)-2-tert-butoxycarbonylamino-propionic acid (37 grams, 195 mmol) in dry tetrahydrofuran (250 mL) at 0° C. was added 4-methyl morpholine (21.5 mL, 195 mmol) followed by isobutylchloroformate (25.3 mL, 195 mmol). The reaction was allowed to warm to ambient temperature and stirred for about two hours. This was followed by the addition of (S)-2-(4-fluoro-benzylamino)-propionic acid methyl ester (34.4 grams, 162 mmol). The resulting mixture was stirred overnight at ambient temperature. The reaction mixture was filtered through a pad of celite and the filter cake was washed with ethyl acetate. The filtrate was concentrated in vacuo, diluted with ethyl acetate and washed with water and brine. The organic layer was dried over magnesium sulfate, filtered and concentrated in vacuo. Chromatography on silica gel gave the title compound (43.2 grams).

Step 3: (3R,6S)-1-(4-Fluoro-benzyl)-3,6-dimethyl-piperazine-2,5-dione

To a solution of (2S)-2-[(2R)-(2-tert-butoxycarbonylamino-propionyl)-(4-fluoro-benzyl)-amino]-propionic acid methyl ester (43 grams, 382 mmol) in dichloromethane (120 mL) at 0° C. was added trifluoroacetic acid (60 mL). The reaction was allowed to warm to ambient temperature and stirred for about 2 hours. The reaction was cooled to 0° C. and slowly quenched by addition of 3 N sodium hydroxide until basic. The resulting mixture was extracted with dichloromethane. The organic layer was dried over magnesium sulfate, filtered and concentrated in vacuo to give the title compound (22 grams).

Step 4: (2R,5S)-1-(4-Fluoro-benzyl)-2,5-dimethyl-piperazine.

To a solution of (3R,6S)-1-(4-fluoro-benzyl)-3,6-dimethyl-piperazine-2,5-dione (22 grams, 87.9 mmol) in dry tetrahydrofuran (160 mL) at 0° C. was added a solution of lithium aluminum hydride (1M in tetrahydrofuran, 373 mL, 373 mmol) dropwise over about 40 minutes. The reaction mixture was then refluxed for about 4 hours, cooled to ambient temperature and slowly quenched with water. The resulting mixture was filtered through a pad of celite and the filter cake was washed with ethyl acetate. The filtrate was then concentrated, diluted with ethyl acetate and washed with saturated aqueous sodium hydrogen carbonate. The organic layer was separated, dried over magnesium sulfate, filtered and concentrated in vacuo to give the title compound (17.7 grams).

Step 5: 2-Chloro-1-[4-(4-fluoro-benzyl)-(2R,5S)-2,5-dimethyl-piperazin-1-yl]-ethanone.

To a solution of (2R,5S)-1-(4-fluoro-benzyl)-2,5-dimethyl-piperazine (2.5 grams, 11.2 mmol) in dry dichloromethane (11 mL) at 0° C. was added triethylamine (1.57 mL, 11.2 mmol) followed by chloroacetyl chloride (0.86 mL, 11.2 mmol). The resulting reaction mixture was stirred for about 30 minutes. The reaction was then filtered through a pad of celite, washed with dichloromethane and the resulting filtrate was concentrated. Chromatography on silica gel gave the title compound (2.84 grams).

Step 6: 5-Chloro-2-{2-[4-(4-fluoro-benzyl)-(2R,5S)-2,5-dimethyl-piperazin-1-yl]-2-oxo-ethoxy}-benzaldehyde.

To a solution of 2-chloro-1-[4-(4-fluoro-benzyl)-(2R,5S)-2,5-dimethyl-piperazin-1-yl]-ethanone (2.87 grams, 9.6 mmol) in dimethylformamide (20 mL) was added 5-chlorosalicylaldehyde (1.65 grams, 10.5 mmol), potassium carbonate (2.64 grams, 19.2 mmol) and potassium iodide (1.59 grams, 9.6 mmol). The resulting mixture was heated to 100° C. for 12 hours. The reaction was cooled, diluted with saturated aqueous brine and extracted with ethyl acetate. The organic layer was dried over magnesium sulfate and filtered. The filtrate was concentrated in vacuo to give crude product. Purification via chromatography on silica gel gave the title compound (3.40 grams).

Step 7: 2-(4-Chloro-2-hydroxymethyl-phenoxy)-1-[4-(4-fluoro-benzyl)-(2R,5S)-2,5-dimethyl-piperazin-1-yl]-ethanone.

To a solution of 5-chloro-2-{2-[4-(4-fluoro-benzyl)-(2R,5S)-2,5-dimethyl-piperazin-1-yl]-2-oxo-ethoxy}-benzaldehyde (0.99 grams, 2.36 mmol) in dry methanol (25 mL) was added sodium borohydride (0.19 grams, 4.92 mmol). After about 1 hour, the reaction was acidified to a pH of about 2 by the addition of 1N hydrochloric acid. After about 5 minutes, the reaction was neutralized with 1N sodium hydroxide and the methanol removed by evaporation. The resulting aqueous suspension was extracted with ethyl acetate. The organic layer was washed with brine, dried over magnesium sulfate, filtered and evaporated to give the title compound (0.98 grams).

Step 8: 2-(4-Chloro-2-chloromethyl-phenoxy)-1-[4-(4-fluoro-benzyl)-(2R,5S)-2,5-dimethyl-piperazin-1-yl]-ethanone.

To 2-(4-chloro-2-hydroxymethyl-phenoxy)-1-[4-(4-fluoro-benzyl)-(2R,5S)-2,5-dimethyl-piperazin-1-yl]-ethanone (0.55 grams, 1.3 mmol) in methylene chloride (6 mL) was added thionyl chloride (0.26 mL, 3.58 mmol). The reaction was heated to reflux for about 2 hours. After cooling, the reaction was quenched by the addition of water. The organic layer was washed with saturated sodium bicarbonate followed by saturated aqueous sodium chloride. The organic layer was then concentrated to afford the title compound as a yellow oil (0.52 grams).

Step 9: (5-Chloro-2-{2-[4-(4-fluoro-benzyl)-(2R,5S)-2,5-dimethyl-piperazin-1-yl]-2-oxo-ethoxy}-benzyl)-phosphonic Acid.

A solution of 2-(4-chloro-2-chloromethyl-phenoxy)-1-[4-(4-fluoro-benzyl)-(2R,5S)-2,5-dimethyl-piperazin-1-yl]-ethanone (0.47 grams, 1.07 mmol) and triethylphosphite (0.22 mL, 1.28 mmol) was stirred at 130° C. for about 12 hours. The reaction was cooled, concentrated and taken directly to the next step. To a solution of (5-chloro-2-{2-[4-(4-fluoro-benzyl)-(2R,5S)-2,5-dimethyl-piperazin-1-yl]-2-oxo-ethoxy}-benzyl)-phosphonic acid diethyl ester (0.57 grams, 1.05 mmol) in dichloromethane (10 mL) at ambient temperature, was added anisole (0.23 mL, 2.10 mmol) and trimethylsilylbromide (0.28 mL, 2.10 mmol). The resulting solution was stirred at ambient temperature for about 3 hours, then quenched with methanol. The reaction mixture was concentrated in vacuo, and the crude product was purified via anion exchange chromatography to give the title compound (0.21 grams, LRMS: 485.1, 483.3).

### EXAMPLE 2

### (5-Bromo-2-{2-[4-4-fluoro-benzyl)-(2R,5S)-2,5-dimethyl-piperazin-1-yl]-2-oxo-ethoxy}-benzyl)-phosphonic Acid

Example 2 was prepared by a method analogous to that described in Example 1. The reaction mixture was concentrated in vacuo, and the crude product was purified via anion exchange chromatography to provide the title compound (LRMS: 530.9).

### EXAMPLE 3

### (5-Bromo-2-{2-[4-(4-fluoro-benzyl)-(2R)-2-methyl-piperazin-1-yl]-2-oxo-ethoxy}-benzyl)-phosphonic Acid

Example 3 was prepared by a method analogous to that described in Example 1. The reaction mixture was concentrated in vacuo, and the crude product was purified via anion exchange chromatography to provide the title compound (LRMS: 516.9).

### EXAMPLE 4

### [2-(5-Chloro-2-{2-[4-(4-fluoro-benzyl)-(2R,5S)-2,5-dimethyl-piperazin-1-yl]-2-oxo-ethoxy}-phenyl)-ethyl]-phosphonic Acid

Step 1: [2-(5-Chloro-2-hydroxy-phenyl)-vinyl]-phosphonic Acid Diethyl Ester.

To a mixture of 5-chloro-2-hydroxy-benzaldehyde (0.65 grams, 4.17 mmol) and (diethoxy-phosphorylmethyl)-phosphonic acid diethyl ester (1.1 mL), was added 50% aqueous NaOH (6 mL). The resulting mixture was stirred at ambient temperature for about 12 hours, then the pH was adjusted to about 3 by careful addition of concentrated hydrochloric acid. The solution was diluted with water and extracted with methylene chloride. The organic layer was dried over magnesium sulfate, filtered and concentrated in vacuo. Chromatography on silica gel gave the title compound (1.21 grams).

Step 2: [2-(5-Chloro-2-hydroxy-phenyl)-ethyl]-phosphonic Acid Diethyl Ester.

To a solution of [2-(5-chloro-2-hydroxy-phenyl)-vinyl]-phosphonic acid diethyl ester (0.50 grams, 1.70 mmol) in ethanol (50 mL) was added calcium carbonate (0.30 grams) and palladium acetate (0.02 grams). The resulting mixture was hydrogenated at 50 psi for about 12 hours. Filtration and concentration in vacuo gave the title compound (0.30 grams).

Step 3: [2-(5-Chloro-2-{2-[4-(4-fluoro-benzyl)-(2R,5S)-2,5-dimethyl-piperazin-1-yl]-2-oxo-ethoxy}-phenyl)-ethyl]-phosphonic Acid Diethyl Ester.

To a solution of 2-chloro-1-[4-(4-fluoro-benzyl)-(2R,5S)-2,5-dimethyl-piperazin-1-yl]-ethanone (0.19 grams, 0.64 mmol) in dimethylformamide (2 mL), was added [2-(5-chloro-2-hydroxy-phenyl)-ethyl]-phosphonic acid diethyl ester (0.21 grams, 0.72 mmol), potassium carbonate (0.24 grams, 1.7 mmol) and potassium iodide (0.10 grams, 0.62 mmol). The mixture was heated to 60° C. for about 12 hours, diluted with brine and extracted with ethyl acetate. The organic layer was dried over magnesium sulfate, filtered and concentrated in vacuo. Chromatography on silica gel gave the title compound (0.28 grams).

Step 4: [2-(5-Chloro-2-{2-[4-(4-fluoro-benzyl)-(2R,5S)-2,5-dimethyl-piperazin-1-yl]-2-oxo-ethoxy}-phenyl)-ethyl]-phosphonic Acid.

To a solution of [2-(5-chloro-2-{2-[4-(4-fluoro-benzyl)-(2R,5S)-2,5-dimethyl-piperazin-1-yl]-2-oxo-ethoxy}-phenyl)-ethyl]-phosphonic acid diethyl ester (0.27 grams, 0.50 mmol) in methylene chloride (5 mL), was added bromotrimethylsilane (0.13 mL, 0.98 mmol) and anisole (0.11 mL, 1.0 mmol) and the resulting solution was stirred for about 3 hours at ambient temperature. Additional bromotrimethylsilane (0.098 mL, 0.74 mmol) and anisole (0.081 mL, 0.74 mmol) were added, and the solution stirred for about an additional 3 hours at ambient temperature. Additional bromotrimethylsilane (0.098 mL, 0.74 mmol) and anisole (0.081 mL, 0.74 mmol) were added and the solution stirred for about an additional 1 hour at ambient temperature. Methanol (5 mL) was then added, and the solution was stirred about 12 hours at ambient temperature. Concentration in vacuo, followed by purification via anion exchange chromatography, gave the title compound (0.21 grams, LRMS: 499.0, 501.1)

### EXAMPLE 5

### (5-Chloro-2-{2-[4-(4-fluoro-benzyl)-(2R,5S)-2,5-dimethyl-piperazin-1-yl]-2-oxo-ethoxy}-benzyl)phosphonic Acid Monoethyl Ester

To a solution of (5-chloro-2-{2-[4-(4-fluoro-benzyl)-(2R,5S)-2,5-dimethyl-piperazin-1-yl]-2-oxo-ethoxy}-benzyl)-phosphonic acid diethyl ester (0.089 grams, 0.165 mmol) in dry dichloromethane (2 mL), was added trimethylsilylbromide (32 μl, 0.242 mmol). The reaction stirred at ambient temperature for about 16 hours. The reaction was quenched with methanol, and the mixture was concentrated in vacuo. Chromatography on silica gel gave the title compound (0.033 grams, LRMS: 513.1).

### EXAMPLE 6

### (5-Chloro-2-{2-[4-(4-fluoro-benzyl)-(2R,5S)-2,5-dimethyl-piperazin-1-yl]-2-oxo-ethoxy}-phenyl)-phosphonic Acid

Step 1: Phosphoric Acid 4-chloro-phenyl Ester Diethyl Ester.

To a solution of 4-chlorophenol (1.0 grams, 7.79 mmol) and triethylamine (0.94 grams, 9.33 mmol) in tetrahydrofuran (26 mL) at 0° C., was added diethylphosphoryl chloride (1.41 grams, 8.17 mmol). The reaction was allowed to slowly warm to ambient temperature and stirred for about 12 hours. The reaction was quenched by addition of water, then extracted with diethyl ether. The organic layer was washed with brine, dried over sodium sulfate, filtered and concentrated in vacuo. Chromatography on silica gel gave the title compound (1.10 grams).

Step 2: (5-Chloro-2-hydroxy-phenyl)-phosphonic Acid Diethyl Ester.

To a solution of n-butyllithium (2.2 mL, 3.78 mmol, 2.5 M in tetrahydrofuran) in tetrahydrofuran (10 mL) at −78° C., was added diisopropyl amine (0.53 mL, 3.78 mmol). After several minutes at −78° C., a solution of phosphoric acid 4-chloro-phenyl ester diethyl ester (0.50 grams, 1.89 mmol) in THF (9 mL) was slowly added. The reaction was stirred at −78° C. for about 1 hour, then warmed to ambient temperature overnight. The reaction was quenched by addition of water then extracted with diethyl ether. The organic layer was dried over sodium sulfate, filtered and concentrated in vacuo. Chromatography on silica gel gave the title compound (0.27 grams).

Step 3: (5-Chloro-2--{2-[4-(4-fluoro-benzyl)-(2R,5S)-2,5-dimethyl-piperazin-1-yl]-2-oxo-ethoxy}-phenyl)-phosphonic Acid Diethyl Ester.

To a solution of 2-chloro-1-[4-(4-fluoro-benzyl)-(2R,5S)-2,5-dimethyl-piperazin-1-yl]-ethanone (0.30 grams, 1.0 mmol) in dimethylformamide (10 mL), was added (5-chloro-2-hydroxy-phenyl)-phosphonic acid diethyl ester (0.26 grams, 1.0 mmol), potassium carbonate (0.28 grams, 2.0 mmol) and potassium iodide (0.17 grams, 1.0 mmol). The mixture was heated to 60° C. for about 12 hours then concentrated in vacuo. The crude product was dissolved in diethyl ether and washed with brine. The organic layer was dried over magnesium sulfate, filtered and concentrated in vacuo. Chromatography on silica gel gave the title compound (0.40 grams).

Step 4: (5-Chloro-2-{2-[4-(4-fluoro-benzyl)-(2R,5S)-2,5-dimethyl-piperazin-1-yl]-2-oxo-ethoxy}-phenyl)-phosphonic Acid.

A solution of (5-chloro-2-{2-[4-(4-fluoro-benzyl)-(2R,5S)-2,5-dimethyl-piperazin-1-yl]-2-oxo-ethoxy}-phenyl)-phosphonic acid diethyl ester (0.090 grams, 0.17 mmol) and bromotrimethylsilane (0.11 mL, 0.85 mmol) in acetonitrile (2 mL) was stirred at ambient temperature for about 12 hours then concentrated in vacuo. Purification via anion exchange chromatography gave the title compound (0.080 grams, LRMS: 471.0, 469.2)

### EXAMPLE 7

### (5-Chloro-2-{2-[4-(4-fluoro-benzyl)-(2R,5S)-2,5-dimethyl-piperazin-1-yl]-2-oxo-ethoxy}-benzyl)-phosphonamidic Acid

Step 1: 2-Benzyloxy-5-chloro-benzaldehyde.

To a solution of 5-chlorosalicylaldehyde (1.0 grams, 6.38 mmol) in dry 4:1 DMF/THF (60 mL), was added potassium carbonate (2.2 grams, 15.9 mmol) and benzyl bromide (1.9 mL, 16.0 mmol). The reaction was stirred for about 16 hours at ambient temperature. The reaction was neutralized with pH=7 buffer and extracted with 1:1 hexanes/diethyl ether. The organic layer was washed with distilled water, brine, and dried over magnesium sulfate and filtered. The filtrate was concentrated in vacuo to give the title compound (2.76 grams).

Step 2: (2-Benzyloxy-5-chloro-phenyl)-methanol.

To a solution of 2-benzyloxy-5-chloro-benzaldehyde (2.75 grams, 11.1 mmol) in dry methanol (100 mL) at 0° C., was added sodium borohydride (0.84 grams, 22.3 mmol). The reaction was slowly warmed to ambient temperature while stirring for about one hour. The reaction was acidified to pH=2 with 1 N hydrochloric acid and diluted with distilled water. The methanol was evaporated from this aqueous solution, and the resulting suspension was extracted with ethyl acetate. The organic layer was washed with brine, dried over magnesium sulfate, filtered, and concentrated in vacuo. Chromatography on silica gel gave the title compound (1.37 grams).

Step 3: 2-Benzyloxy-5-chloro-benzyl Chloride.

To a solution of (2-benzyloxy-5-chloro-phenyl)-methanol (1.37 grams, 5.51 mmol) in dry dichloromethane (60 mL) was added thionyl chloride (0.8 mL, 11.0 mmol). The reaction was stirred at ambient temperature for about 16 hours. The reaction was quenched with a saturated sodium bicarbonate solution and extracted with dichloromethane. The organic layer was washed with brine, dried over magnesium sulfate, filtered, and concentrated in vacuo to give the title compound (1.43 grams).

Step 4: (2-Benzyloxy-5-chloro-benzyl)-phosphonic Acid Diethyl Ester.

A solution of 2-benzyloxy-5-chloro-benzyl chloride (0.40 grams, 1.50 mmol) and triethylphosphite (0.3 mL, 1.75 mmol) was stirred at 100° C. for about 19 hours. Chromatography on silica gel of the crude reaction mixture gave the title compound (0.35 grams).

Step 5: (2-Benzyloxy-5-chloro-benzyl)-phosphonamidic Acid Monoethyl Ester.

First, to a solution of (2-benzyloxy-5-chloro-benzyl)-phosphonic acid diethyl ester (0.24 grams, 0.65 mmol) in anhydrous toluene (6 mL), was added PCl₅ (0.40 grams, 1.94 mmol). The reaction was stirred at 80° C. for about 15 hours. The reaction was cooled, concentrated. Second, the crude chloro intermediate was cooled to −78° C. followed by addition of ethanol. Ammonia was then condensed into this solution at −78° C. The reaction slowly warmed to ambient temperature while stirring for about 1 hour. The reaction was concentrated in vacuo and silica gel chromatography yielded the title compound (0.15 grams).

Alternatively, the above second step of Step 5 can be accomplished by adding an ethanolic ammonia solution to the crude chloro intermediate at −45° C.

Step 6: (5-Chloro-2-hydroxy-benzyl)-phosphonamidic Acid Monoethyl Ester.

To a solution of (2-benzyloxy-5-chloro-benzyl)-phosphonamidic acid monoethyl ester (0.15 grams, 0.44 mmol) in ethanol (20 mL) was added 10% palladium on activated carbon (30 mg). This suspension was placed under 48 psi of hydrogen gas and shaken at ambient temperature for about 1.5 hours. The reaction was filtered through a pad of celite, and the filter cake was washed with methanol. The combined filtrate and wash was concentrated in vacuo. Chromatography on silica gel yielded the title compound (0.12 grams).

Step 7: (5-Chloro-2-{2-[4-(4-fluoro-benzyl)-2,5-dimethyl-piperazin-1-yl]-2-oxo-ethoxy}-benzyl)-phosphonamidic Acid Monoethyl Ester.

To a solution of (5-chloro-2-hydroxy-benzyl)-phosphonamidic acid monoethyl ester (0.032 grams, 0.12 mmol), 1-[4-(4-fluoro-benzyl)-(2R,5S)-2,5-dimethyl-piperazin-1-yl]-2-hydroxy-ethanone (0.040 grams, 0.16 mmol), and triphenylphosphine (0.042 grams, 0.16 mmol) in anhydrous toluene (2 mL), was dropwise added diethyl azodicarboxylate (25 μl, 0.16 mmol). The reaction was stirred at ambient temperature for about 17 hours. The reaction was neutralized with pH=7 buffer and extracted with ethyl acetate. The organic layer was washed with brine, dried over magnesium sulfate, filtered, and concentrated in vacuo. Chromatography on silica gel yielded the title compound (0.047 grams).

Step 8: (5-Chloro-2-{2-[4-(4-fluoro-benzyl)-(2R,5S)-2,5-dimethyl-piperazin-1-yl]-2-oxo-ethoxy}-benzyl)-phosphonamidic Acid

To a solution of (5-chloro-2-{2-[4-(4-fluoro-benzyl)-2,5-dimethyl-piperazin-1-yl]-2-oxo-ethoxy}-benzyl)-phosphonamidic acid monoethyl ester (0.025 grams, 0.05 mmol) in dry dichloromethane (1 mL), was added trimethylsilylbromide (10 μl, 0.08 mmol). The reaction was stirred at ambient temperature for about 3 hours. Additional trimethylsilylbromide (20 μl, 0.15 mmol) was added to the reaction, and the reaction continued to stir at ambient temperature for about 20 hours. The reaction was quenched with methanol and the mixture concentrated in vacuo. Chromatography on silica gel gave the title compound in quantitative yield. (LRMS: 485.0)

### Example 8

### (5-Chloro-2-{2-[4-(4-fluoro-benzyl)-(2R,5S)-2,5-dimethyl-piperazin-1-yl]-2-oxo-ethoxy}-benzyl)-methyl-phosphinic Acid

A solution of 2-(4-chloro-2-chloromethyl-phenoxy)-1-[4-(4-fluoro-benzyl)-(2R,5S)-2,5-dimethyl-piperazin-1-yl]-ethanone (0.104 grams, 0.24 mmol) and methyl diethoxyphosphine (0.050 mL, 0.33 mmol) was stirred at 130° C. for about 15 hours. The reaction was cooled and concentrated to give 0.11 grams of crude (5-chloro-2-{2-[4-(4-fluoro-benzyl)-(2R,5S)-2,5-dimethyl-piperazin-1-yl]-2-oxo-ethoxy}-benzyl)-methyl-phosphinic acid ethyl ester, which was taken directly to the next step. To a solution of (5-chloro-2-{2-[4-(4-fluoro-benzyl)-(2R,5S)-2,5-dimethyl-piperazin-1-yl]-2-oxo-ethoxy}-benzyl)-methyl-phosphinic acid ethyl ester (0.043 grams, 0.084 mmol) in dichloromethane (1 mL) at ambient temperature, was added trimethylsilylbromide (0.020 mL, 0.15 mmol). The resulting solution was stirred at ambient temperature for about 15 hours, then additional trimethylsilylbromide (0.020 mL, 0.15 mmol) was added and the reaction stirred for about an additional 4 hours, then quenched with methanol. The reaction mixture was concentrated in vacuo, and the crude product was purified via flash chromatography on silica gel to give the title compound (0.015 grams, LRMS: 483.1, 481.3).

1. A compound of the Formula I, 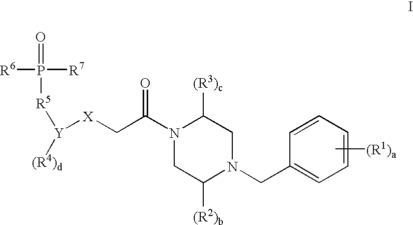
a prodrug thereof, or a pharmaceutically acceptable salt of the compound or the prodrug thereof; wherein,
a=0, 1, 2, 3, 4 or 5;
b=0, 1 or 2;
c=0, 1 or 2;
d=0, 1, 2, 3 or −4;
X is O, S, CH₂ or NR⁶;
Y is (C₆-C₁₀)aryl or (C₂-C₉)heteroaryl;
each R¹ is independently: hydroxy, halo, (C₁-C₈)alkyl optionally substituted with 1 to 3 fluorine atoms, (C₁-C₈)alkoxy optionally substituted with 1-3 fluorine atoms, HO(C₁-C₈)alkyl-, cyano, amino, H₂N(C₁-C₈)alkyl-, carboxy, acyl, (C₁-C₈)alkyl(C═O)(C₁-C₈)alkyl-, H₂N(C═O)—, or H₂N(C═O)(C₁-C₈)alkyl-;
each R² and R³ are independently: oxo, (C₁-C₈)alkyl optionally substituted with 1-3 fluorine atoms, (C₃-C₈)cycloalkyl-, (C₃-C₈)cycloalkyl-(C₁-C₈)alkyl-, (C₆-C₁₀)aryl-, (C₆-C₁₀)aryl(C₁-C₈)alkyl-, HO(C₁-C₈)alkyl-, (C₁-C₈)alkyl-O—(C₁-C₈)alkyl-, H₂N(C₁-C₈)alkyl-, (C₁-C₈)alkyl-NH—(C₁-C₈)alkyl-, [(C₁-C₈)alkyl]₂N—(C₁-C₈)alkyl-, (C₂-C₉)heterocyclyl(C₁-C₈)alkyl-, (C₁-C₈)alkyl(C═O)NH(C₁-C₈)alkyl-, (C₁-C₈)alkyl-O—(C═O) NH(C₁-C₈)alkyl-, H₂N(C═O)NH(C₁-C₈)alkyl-, (C₁-C₈)alkyl-SO₂—NH(C₁-C₈)alkyl-, (C₂-C₉)heteroaryl(C₁-C₈)alkyl-, H₂N(C═O), or H₂N(C═O)(C₁-C₈)alkyl-;
each R⁴ is independently: HO—, halo-, NC—, HO(C═O)—, H₂N—, (C₁-C₈)alkylNH—, [(C₁-C₈)alkyl]₂N—, (C₁-C₈)alkyl-, optionally substituted with 1-3 fluorine atoms, (C₁-C₈)alkoxy optionally substituted with 1-3 fluorine atoms, HO(C₁-C₈)alkyl-, (C₁-C₈)alkyl-O—(C₁-C₈)alkyl-, H₂N(C₁-C₈)alkyl—, (C₁-C₈)alkylNH(C₁-C₈)alkyl-, [(C₁-C₈)alkyl]₂N(C₁-C₈)alkyl-, (C₁-C₈)alkyl(C═O)—, (C₁-C₈)alkyl(C═O)(C₁-C₈)alkyl-, (C₆-C₁₀)aryl-, (C₂-C₉)heteroaryl-, (C₆-C₁₀)aryloxy-, H₂N(C═O)—, H₂N(C═O)(C₁-C₈)alkyl-, (C₁0C₈)alkylNH(C═O)—, (C₁-C₈)alkyl-NH(C═O)(C₁-C₈)alkyl-, [(C₁-C₈)alkyl]₂N(C═O)—, [(C₁-C₈)alkyl]₂N(C═O)(C₁-C₈)alkyl-, (C₃-C₈)cycloalkyl-, (C₁-C₈)alkylSO₂—, NC(C₁-C₈)alkyl-, (C₁-C₈)alkyl(C═O)NH—, H₂N(C═O)NH— or H₂N(C═O)NH(C₁-C₈)alkyl-;
R⁵ is a bond or a (C₁-C₈)alkyl-;
R⁶ is independently: hydroxy, amine or (C₁-C₈)alkyl-NH—; and
R⁷ is independently: hydrogen, hydroxyl, (C₁-C₈)alkoxy- or (C₁-C₈)alkyl-.

2. A compound according to claim 1, wherein the compound of Formula I has the stereochemistry showin in Formula Ia 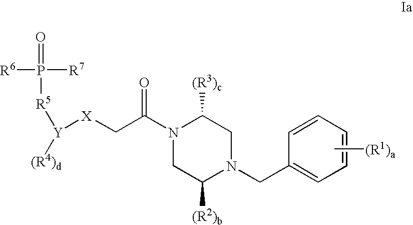
wherein a, b, c, X, Y, R¹, R², R³, R⁴, R⁵, R⁶ and R⁷ are as described above.

3. A compound according to claim 1, wherein R¹ is: hydroxy, halo, cyano, (C₁-C₈)alkyl-optionally substituted with 1-3 fluorine atoms, or (C₁-C₈)alkoxy optionally substituted with 1-3 fluorine atoms.

4. A compound according to claim 1, wherein R⁴ is hydroxyl, cyano, (C₁-C₈)alkyl-optionally substituted with 1-3 fluorine atoms, (C₁-C₈)alkoxy optionally substituted with 1-3 fluorine atoms, (C₁-C₈)alkyl(C═O)— or halo-.

5. A compound according to claim 1, wherein X is O and R⁵ is (C₁-C₃)alkyl-.

6. A compound according to claim 1, wherein R² and R³ are each independently: (C₁-C₈)alkyl-, optionally substituted with 1-3 fluorine atoms; (C₃-C₈)cycloalkyl-; (C₃-C₈)cycloalkyl-(C₁-C₈)alkyl-; (C₆-C₁₀)aryl-; (C₆-C₁₀)aryl(C₁-C₈)alkyl-; HO(C₁-C₈)alkyl-; H₂N(C₁-C₈)alkyl-; (C₂-C₉)heterocyclyl(C₁-C₈)alkyl-; (C₁-C₈)alkyl-O—(C═O)NH(C₁-C₈)alkyl-; H₂N(C═O)NH(C₁-C₈)alkyl-; (C₁-C₈)alkyl-SO₂NH(C₁-C₈)alkyl-; (C₂-C₉)heteroaryl(C₁-C₈)alkyl-; H₂N(C═O)— or H₂N(C═O)(C₁-C₈)alkyl-.

7. A compound according to claim 6, wherein R² and R³ are each independently (C₁-C₈)alkyl-, optionally substituted with 1-3 fluorine atoms; or (C₃-C₈)cycloalkyl-.

8. A compound according to claim 1, wherein
R¹ is: hydroxy, halo, cynao, (C₁-C₈)alkyl optionally substituted with 1-3 fluorine atoms, or (C₁-C₈)alkoxy-optionally substituted with 1-3 fluorine atoms;
R² and R³ are each independently (C₁-C₈)alkyl, optionally substituted with 1-3 fluorine atoms; or (C₃-C₈)cycloalkyl-;
R⁴ is HO—, NC—, (C₁-C₈)alkyl-optionally substituted with 1-3 fluorine atoms, (C₁-C₈)alkoxy optionally substituted with 1-3 fluorine atoms, (C₁-C₈)alkyl(C═O)— or halo-;
X is O; and
R⁵ is (C₁-C₃)alkyl-.

9. A compound according to claim 1, wherein the compound is:
(5-Chloro-2-{2-[4-(4-fluoro-benzyl)-(2R,5S)-2,5-dimethyl-piperazin-1-yl]-2-oxo-ethoxy}-benzyl)-phosphonic acid;
(5-Chloro-2-{2-[4-(4-fluoro-benzyl)-(2R)-2-methyl-piperazin-1-yl]-2-oxo-ethoxy}-benzyl)-phosphonic acid;
(5-Chloro-2-{2-[(2R)-2-ethyl-4-(4-fluoro-benzyl)-piperazin-1-yl]-2-oxo-ethoxy}-benzyl)-phosphonic acid;
(5-Bromo-2-{2-[4-(4-fluoro-benzyl)-(2R,5S)-2,5-dimethyl-piperazin-1-yl]-2-oxo-ethoxy}-benzyl)-phosphonic acid;
(5-Bromo-2-{2-[4-(4-fluoro-benzyl)-(2R)-2-methyl-piperazin-1-yl]-2-oxo-ethoxy}-benzyl)-phosphonic acid;
[2-(5-Chloro-2-{2-[4-(4-fluoro-benzyl)-(2R,5S)-2,5-dimethyl-piperazin-1-yl]-2-oxo-ethoxy}-phenyl)-ethyl]-phosphonic acid;
[2-(5-Chloro-2-{2-[4-(4-fluoro-benzyl)-(2R)-2-methyl-piperazin-1-yl]-2-oxo-ethoxy}-phenyl)-ethyl]-phosphonic acid;
[2-(5-Bromo-2-{2-[4-(4-fluoro-benzyl)-(2R,5S)-2,5-dimethyl-piperazin-1-yl]-2-oxo-ethoxy}-phenyl)-ethyl]-phosphonic acid;
[2-(5-Bromo-2-{2-[4-(4-fluoro-benzyl)-(2R)-2-methyl-piperazin-1-yl]-2-oxo-ethoxy}phenyl)-ethyl]-phosphonic acid;
(5-Chloro-2-{2-[4-(4-chloro-benzyl)-(2R,5S)-2,5-dimethyl-piperazin-1-yl]-2-oxo-ethoxy}benzyl)-phosphonic acid;
(5-Chloro-2-{2-[4-(4-chloro-benzyl)-(2R)-2-methyl-piperazin-1-yl]-2-oxo-ethoxy}-benzyl)-phosphonic acid;
(5-Bromo-2-{2-[4-(4-chloro-benzyl)-(2R,5S)-2,5-dimethyl-piperazin-1-yl]-2-oxo-ethoxy}-benzyl)-phosphonic acid;
(5-Bromo-2-{2-[4-(4-chloro-benzyl)-(2R)-2-methyl-piperazin-1-yl]-2-oxo-ethoxy}-benzyl)-phosphonic acid;
(5-Chloro-2-{2-[4-(3,4-difluoro-benzyl)-(2R,5S)-2,5-dimethyl-piperazin-1-yl]-2-oxo-ethoxy}-benzyl)-phosphonic acid;
(5-Chloro-2-{2-[4-(3,4-difluoro-benzyl)-(2R)-2-methyl-piperazin-1-yl]-2-oxo-ethoxy}-benzyl)-phosphonic acid;
(5-Bromo-2-{2-[4-(3,4-difluoro-benzyl)-(2R,5S)-2,5-dimethyl-piperazin-1-yl]-2-oxo-ethoxy}-benzyl)-phosphonic acid;
(5-Bromo-2-{2-[4-(3,4-difluoro-benzyl)-(2R)-2-methyl-piperazin-1-yl]-2-oxo-ethoxy}-benzyl)-phosphonic acid;
[2-(5-Chloro-2-{2-[4-(4-chloro-benzyl)-(2R,5S)-2,5-dimethyl-piperazin-1-yl]-2-oxo-ethoxy}-phenyl)-ethyl]-phosphonic acid;
[2-(5-Bromo-2-{2-[4-(4-chloro-benzyl)-(2R,5S)-2,5-dimethyl-piperazin-1-yl]-2-oxo-ethoxy}-phenyl)-ethyl]-phosphonic acid;
[2-(5-Chloro-2-{2-[4-(3,4-difluoro-benzyl)-(2R,5S)-2,5-dimethyl-piperazin-1-yl]-2-oxo-ethoxy}-phenyl)-ethyl]-phosphonic acid;
[2-(5-Bromo-2-{2-[4-(3,4-difluoro-benzyl)-(2R,5S)-2,5-dimethyl-piperazin-1-yl]-2-oxo-ethoxy}-phenyl)-ethyl]-phosphonic acid;
(5-Chloro-2-{2-[4-(4-fluoro-benzyl)-(2R,5S)-2,5-dimethyl-piperazin-1-yl]-2-oxo-ethoxy}-pyridin-3-ylmethyl)-phosphonic acid;
(5-Bromo-2-{2-[4-(4-fluoro-benzyl)-(2R,5S)-2,5-dimethyl-piperazin-1-yl]-2-oxo-ethoxy}-pyridin-3-ylmethyl)-phosphonic acid;
[2-(5-Chloro-2-{2-[4-(4-fluoro-benzyl)-(2R,5S)-2,5-dimethyl-piperazin-1-yl]-2-oxo-ethoxy}-pyridin-3-yl)-ethyl]-phosphonic acid;
[2-(5-Bromo-2-{2-[4-(4-fluoro-benzyl)-(2R,5S)-2,5-dimethyl-piperazin-1-yl]-2-oxo-ethoxy}pyridin-3-yl)-ethyl]-phosphonic acid;
(5-Chloro-2-{2-[4-(4-fluoro-benzyl)-(2R,5S)-2,5-dimethyl-piperazin-1-yl]-2-oxo-ethoxy}benzyl)-phosphinic acid;
(5-Chloro-2-{2-[4-(4-fluoro-benzyl)-(2R,5S)-2,5-dimethyl-piperazin-1-yl]-2-oxo-ethoxy}benzyl)-methyl-phosphinic acid;
(5-Chloro-2-{2-[4-(4-fluoro-benzyl)-(2R,5S)-2,5-dimethyl-piperazin-1-yl]-2-oxo-ethoxy}-benzyl)-ethyl-phosphinic acid;
(5-Chloro-2-{2-[4-(4-fluoro-benzyl)-(2R,5S)-2,5-dimethyl-piperazin-1-yl]-2-oxo-ethoxy}-benzyl)-phosphonic acid monomethyl ester;
(5-Chloro-2-{2-[4-(4-fluoro-benzyl)-(2R,5S)-2,5-dimethyl-piperazin-1-yl]-2-oxo-ethoxy}-benzyl)-phosphonic acid monoethyl ester;
(5-Chloro-2-{2-[4-(4-fluoro-benzyl)-(2R,5S)-2,5-dimethyl-piperazin-1-yl]-2-oxo-ethoxy}-benzyl)-ethyl-phosphonamidic acid;
(5-Chloro-2-{2-[4-(4-fluoro-benzyl)-(2R,5S)-2,5-dimethyl-piperazin-1-yl]-2-oxo-ethoxy}-benzyl)-phosphonamidic acid monomethyl ester; or
(5-Chloro-2-{2-[4-(4-fluoro-benzyl)-(2R,5S)-2,5-dimethyl-piperazin-1-yl]-2-oxo-ethoxy}-benzyl)-phosphonamidic acid monoethyl ester.

10. A pharmaceutical composition comprising a therapeutically effective amount of a compound according to claim 1, a prodrug thereof or a pharmaceutically acceptable salt of the compound or the prodrug, and a pharmaceutically acceptable diluent or carrier.

11. A therapeutic method of inhibiting MIP-1α and/or RANTES from binding to the receptor CCR1 in a mammal, including a human, comprising administering to a mammal in need of such treatment a therapeutically effective amount of a compound according to claim 1.

12. A method of treating a condition mediated by inhibiting MIP-1α and/or RANTES from binding to the receptor CCR1, comprising admininistering to a mammal in need of such treatment a therapeutically effective amount of a compound according to claim 1.

13. The method according to claim 12, wherein the condition treated or prevented is selected from autoimmune diseases; fibrosis; allergic conditions; acute and chronic lung inflammation; atherosclerosis; Alzheimer's disease; vascular inflammation resulting from tissue transplant or during restenosis; acute and chronic inflammatory conditions; acute or chronic transplant rejection; HIV infectivity; granulomatous diseases; conditions associated with leptin production; sequelae associated with cancer; tissue damage caused by inflammation induced by infectious agents; viral inflammation of the lung or liver; gastrointestinal inflammation; inflammation resulting from bacterial meningitis, HIV-1, HIV-2, HIV-3, cytomegalovirus, adenoviruses, Herpes viruses, fungal meningitis, lyme disease, or malaria; rheumatoid arthritis; Takayasu arthritis; psoriatic arthritis; ankylosing spondylitis; type I diabetes (recent onset); lupus; inflammatory bowel disease; Chrohn's disease; optic neuritis; psoriasis; multiple sclerosis; polymyalgia rheumatica; uveitis; thyroiditis; vasculitis; pulmonary fibrosis; idiopathic pulmonary fibrosis; interstitial pulmonary fibrosis; fibrosis associated with end-stage renal disease; fibrosis caused by radiation; tubulointerstitial fibrosis; subepithelial fibrosis; scleroderma; progressive systemic sclerosis; hepatic fibrosis; primary and secondary biliary cirrhosis; asthma; contact dermatitis; atopic dermatitis; chronic bronchitis; chronic obstructive pulmonary disease; adult Respiratory Distress Syndrome; Respiratory Distress Syndrome of infancy; immune complex alveolitis; restenosis following angioplasty and/or stent insertion; synovial inflammation caused by arthroscopy, hyperuremia, or trauma; osteoarthritis; ischemia reperfusion injury; glomerulonephritis; nasal polyosis; enteritis; Behcet's disease; preeclampsia; oral lichen planus; Guillian-Barre syndrome; xeno-transplantation rejection; sarcoidosis; leprosy; tuberculosis; obesity; cachexia; anorexia; type II diabetes; hyperlipidemia; hypergonadism; sequelae associated with multiple myeloma; viral-induced encephalomyelitis or demyelination; viral inflammation of the lung or liver caused by influenza or hepatitis; and H. pylori infection.

14. A therapeutic method of treating a condition mediated by inhibiting the production of metalloproteinases and cytokines at inflammatory sites comprising administering to a mammal, including a human, in need of such treatment a therapeutically effective a mount of a compound according to claim 1.

15. The method according to claim 14, wherein the condition treated is joint tissue damage, hyperplasia, pannus formation, bone resorption, hepatic failure, Kawasaki syndrome, myocardial infarction, acute liver failure, septic shock, congestive heart failure, pulmonary emphysema or dyspnea associated therewith.
